(19) United States
(12) Patent Application Publication
(10) Pub. No.: US 2006/0009382 A1
(43) Pub. Date: Jan. 12, 2006

(54) Use of a CD28 binding pharmaceutical substance for making a pharmaceutical composition with dose-dependent effect

(76) Inventors: Thomas Hanke, Wurzburg (DE); Chia-Huey Lin, Wurzburg (DE)

Correspondence Address: MAYER, FORTKORT & WILLIAMS, PC, 251 NORTH AVENUE WEST, 2ND FLOOR, WESTFIELD, NJ, 07090, US

(21) Appl. No.: 10/946,836

(22) Filed: Sep. 22, 2004

(30) Foreign Application Priority Data
Sep. 22, 2003 (DE) 103 45 008.4
Oct. 20, 2003 (DE) 103 49 371.9

(51) Int. Cl. A61K 38/17; A61K 39/395

(52) U.S. Cl. 514/12; 424/144.1

(57) ABSTRACT

The invention relates to the use of a CD28-specific superagonistic monoclonal antibody (MAB) or of a mimetic compound of the same, for producing a pharmaceutical composition, wherein the dosage is below or above a defined dosage limit.

### FIELD OF THE INVENTION

The invention relates to the use of a CD28 binding pharmaceutical substance for making a pharmaceutical composition.

### BACKGROUND OF THE INVENTION AND PRIOR ART

Autoimmunity occurs, when the adaptive immune system composed of B and T lymphocytes (B and T cells) deems autologous structures (“within itself”) heter-ologous and mobilizes its effector mechanisms against them. Multiple sclerosis, type 1 diabetes and rheumatoid arthritis (RA) are examples of autoimmune diseases, where it is assumed that self-reactive T lymphocytes present in the body play a central role for the pathogenesis of the disease (Ermann and Fathman, 2001).

Normally, T cells are tolerant with regard to autologous tissue and only react on the main histocompatibilty complex (MHC)-mediated detection of heterologous structures with an activation and effector functions. This can be explained by different “tolerance-inducing” mechanisms: 1) Negative selection: during their maturation in the thymus, such T cells are elininated, the antigen receptors of which have a high affinity for autoantigens and are thus potentially autoaggressive and pathogenic; 2) Anergy induction: in case of an incomplete stimulation of T cells, e.g. by lacking costimulation in secondary lymphoid organs, autoreactive T cells can be brought in a state of temporary functional passivity or anergy; 3) Active suppression: autoreactive and potentially pathogenic T cells are actively prevented by a specialized T cell subpopulation, so-called regulatory T cells (Tregs, formerly also designated “suppressor cells”) from performing effector functions.

The great importance of this mechanism for maintaining immunologic tolerance is summarized in a multitude of recent survey articles (Maloy and Powrie, 2001; Sakaguchi et al., 2001; Shevach, 2002).

The essential features of the regulatory T cells can be described as follows: i) they are CD4 T lymphocytes having a wide repertoire of T cell antigen receptors (TCR) of the conventional α/β type. Regulatory T lymphocytes maturate in the thymus and are generally characterized in the peripheral lymphoid organs by expression of the low-molecular isoform of the CD45 molecule, the receptors CD25 (IL-2R alpha chain), CD152 (CTLA-4), glucocorticoid-induced TNF receptor family-related protein (GITR), and the transcription factor foxp3 (Khattri et al., 2003). By combined analysis of these markers, a substantial delimitation or regulatory T cells from the remainder of the CD4 T cell population is

By elimination of regulatory T lymphocytes in vivo on the one hand and adoptive cell transfer experiments on the other hand it could be shown that regulatory T lymphocytes are important for the prevent ion of organ-specific autoimmune-inflammatory diseases, such as the colitis, the insulitis or the orchitis. The ratio in numbers of the regulatory T cells and conventional T cells, which can become pathogenic T cells because of the lacking of Tregs, seems to play an important role (Sakaguchi et al., 1995).

The pharmaceutical mechanism of regulatory T cells is not yet fully clear. In vivo, not however in cell culture, the anti-inflammatory cytokines IL-10 and TGFβ seem to play an important role (Maloy and Powrie, 2001). From cell culture experiments, on the other hand, it clearly results that a direct contact between the regulatory T lymphocytes and the T lymphocytes to be suppressed is essential; possibly CTLA-4 is here functionally important (Thornton and Shevach, 1998). Even without the not yet completely clarified way of functioning of regulatory T lymphocytes, it can definitely be seen that here a general principle of maintaining the immunologic self-tolerance is effective, which can be made useful for the specific control of autoimmunity.

The aim of a pharmacological activation of regulatory T cells for the therapy of autoimmune diseases is to excite the multiplication and function of these cells in the organism, without the cells losing their suppressant function. This approach has proven up to now to be extremely difficult. The activation of regulatory T cells, in the following also called Tregs, via the TCR will, even in combination with a conventional signal via the costimulatory molecule CD28, not normally lead to a proliferation of these cells.

In the rat animal model, it could be shown that a novel monoclonal antibody (MAB) with specificity for the CD28 molecule—JJ316—efficiently activates T lymphocytes in vitro as well as in vivo without TCR stimulation (Tacke et al., 1997), i.e. acts “superagonistically”. This antibody—in spite of its strong T cell-stimulatory properties—is very well tolerated in vivo, in contrast to all other known T cellactivating substances (Rodriguez-Palmero et al., 1999; Tacke et al., 1997). Newest results show that with in vivo administration of JJ316 functional Tregs in the rat are overproportionally expanded (Lin and Hunig, 2003). This preferential stimulation of regulatory T cells with a CD4+CD25+CDTLA-4+ phenotype is most probably the reason for the fair tolerability and the anti-inflammatory effect of the antibody in the organism (see also DE 197 22 888; PCT/DE03/00890).

As a new approach for the multiplication of Tregs for therapeutic purposes in man, a superagonistic antibody for the human CD28 molecule, called CD28-SuperMAB, has been developed. These MABs very efficiently excite in vitro T lymphocytes of man to cell division (Luhder et al., 2003). First investigations indicate that, similar to the rat, the CD4+CD25+ regulatory cells present in a low number in the peripheral blood of man can only difficultly be multiplied by costimulation, i.e. by anti-TCR plus conventional anti-CD28 stimulation, but very well by superagonistic CD28 stimulation (PCT/DE03/00890). Furthermore, it could be shown in a pilot study that an in vitro administration of anti-human CD28-SuperMAB induces in a rhesus monkey in vivo a profound activation of T cells, without clinically visible side effects. This speaks for that the human CD28-SuperMAB also may have an anti-phlogistic effect. Summarizing the above, the tests showed that the CD28-SuperMAB in vitro activated human Tregs and in vivo in an animal model is very well tolerated, in spite of an induction of a T cell activation.

Literature cited above:

Barnes, D. A., et al., J. Clin. Invest. 101: 2910-2919, 1998; Ermann, J., et al., Nat. Immunol. 2:759-761, 2001; Khattri, R., et al., Nat. Immunol. 4:337-342, 2003; Lin, C. H., et al., Eur. J. Immunol. 33:626-638, 2003; Luhder, F., et al., J. Exp. Med. 197:955-966, 2003; Maloy, K. J., et al., Nat. Immunol. 2:816-822, 2001; Rodriguez-Palmero, M., et al., Eur. J. Immunol. 29:3914-3924, 1999.

### TECHNICAL OBJECT OF THE INVENTION

The invention is based on the technical object to specify a pharmaceutical composition, which can be used as an anti-inflammatory as well as immune-reconstituting substance.

### BASICS OF THE INVENTION AND PREFERRED EMBODIMENTS

For achieving the above technical object, the invention teaches the use of a CD28-specific superagonistic monoclonal antibody (MAB) or of a mimetic compound of the same, for producing a pharmaceutical composition for the treatment or prophylaxis of autoimmune-caused inflammatory diseases or for the immune reconstitution, wherein the pharmaceutical composition is prepared such that the dosis of the MAB to be administered is below a defined first dosis limit, if the treatment or prophylaxis of an autoimmune-caused inflammatory disease is indicated, and that the dosis of the MAB to be administered is above a defined second dosis limit, if an immune reconstitution is indicated.

The invention is based on a series of new experiments and findings therefrom. Such experiments have shown that in the rat model, low doses of JJ316 (superagonistic CD28-specific MAB) expand Tregs to a stronger degree than conventional T cells. Further, a low dosis has in a model for rheumatoid arthritis the same therapeutic effect as a 5 times higher dosis. The basic concept of the invention is therefor to be capable to separate the anti-inflammatory effect of the antibody at a low dosis from the generally T cell-stimulating/immune-reconstituting effect at a high dosis. In other words, a pharmaceutical composition is optionally prepared with a dosis of the administration units and/or a dosis instruction, which depending on the desired indication (anti-inflammatory/immune-reconstituting) is below or above the respective defined dosis limits.

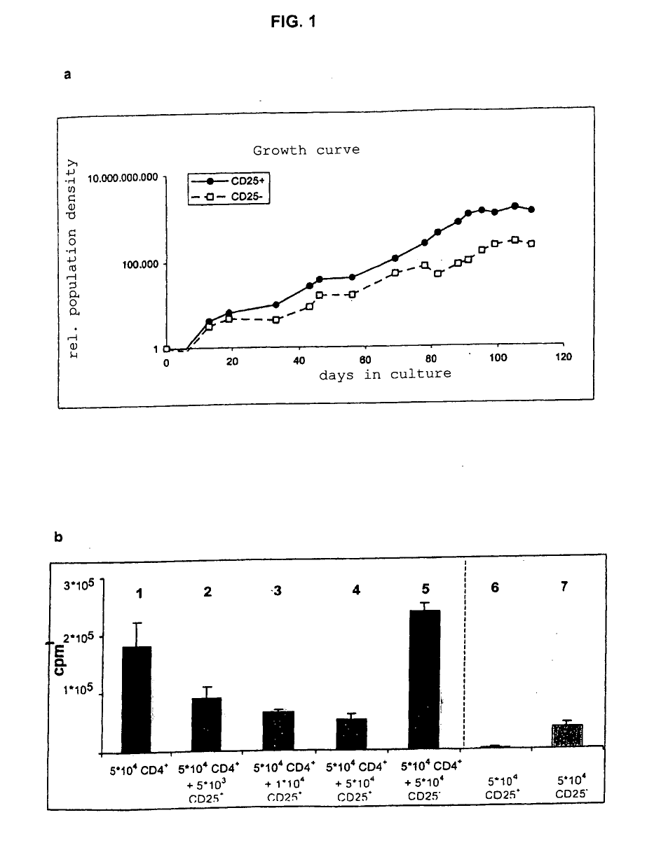

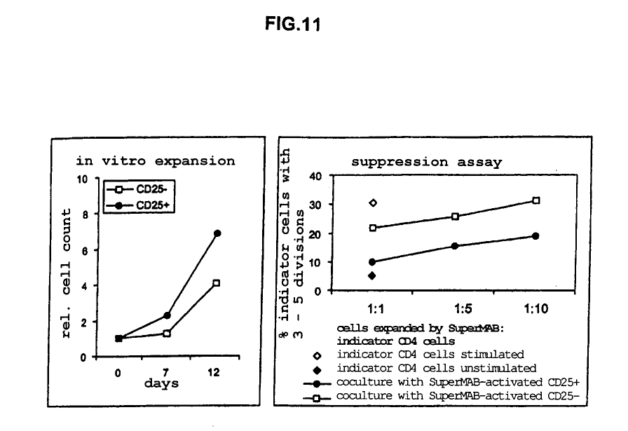

As an aspect of independent importance, it was found that a superagonistic anti-rat or anti-human CD28 antibody permits a long-term culture of functional Tregs in vitro (see also FIGS. 1 and 11), and that therefrom the possibility of a selective expansion of Tregs in vitro and of a therapeutic re-infusion for suppressing autoimmune diseases is generated. Insofar, another subject matter of the invention is a method for the in vitro expansion of Tregs, wherein Tregs are incubated with MABs according to the invention and expanded over a period of time of at least 3 to 10 days, preferably by at least the factor 2 to 4 (number of Tregs after the expansion related to number of Tregs before the expansion).

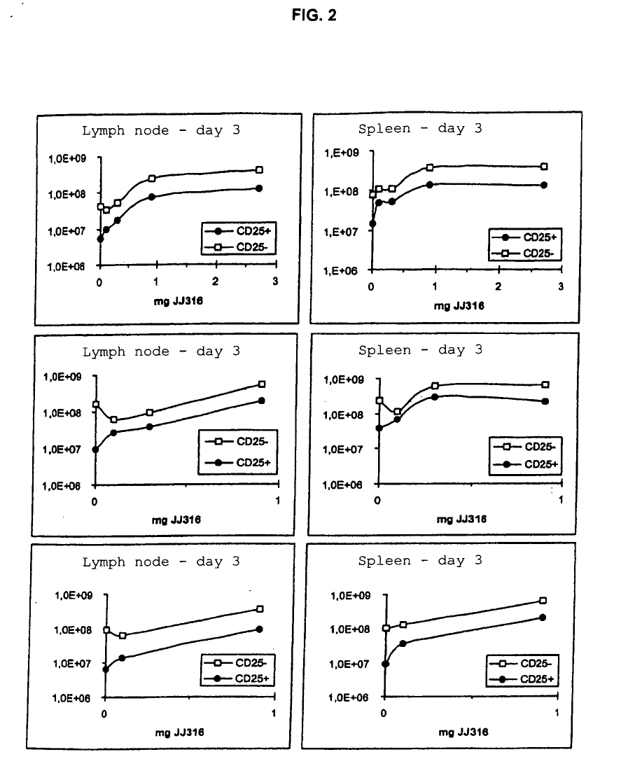

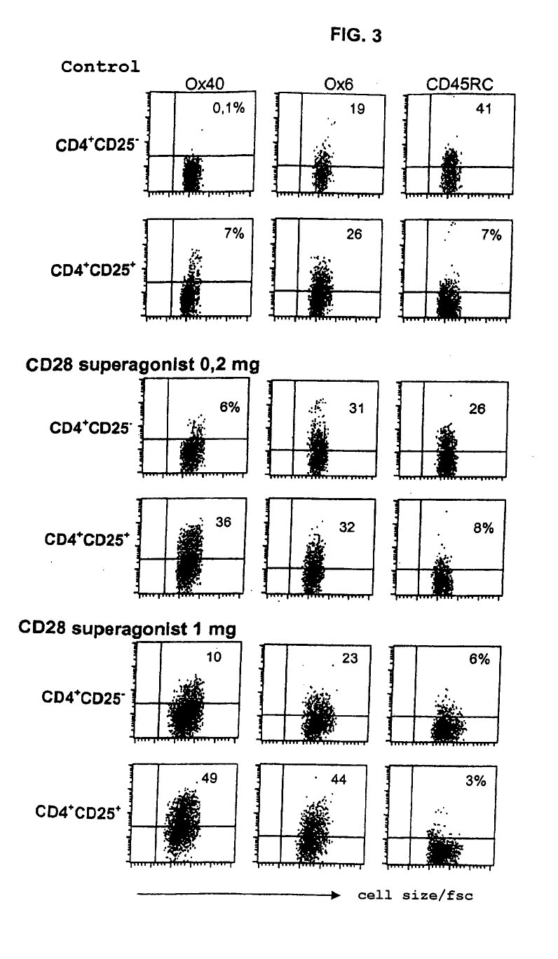

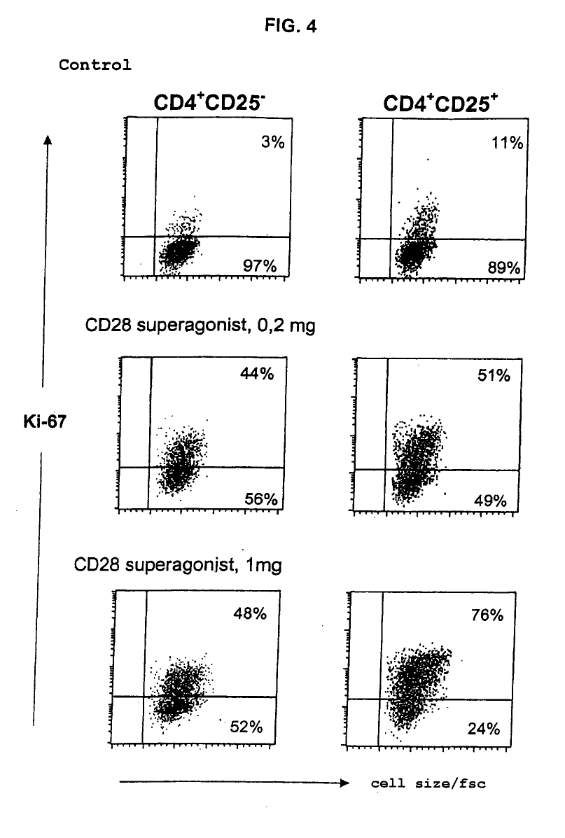

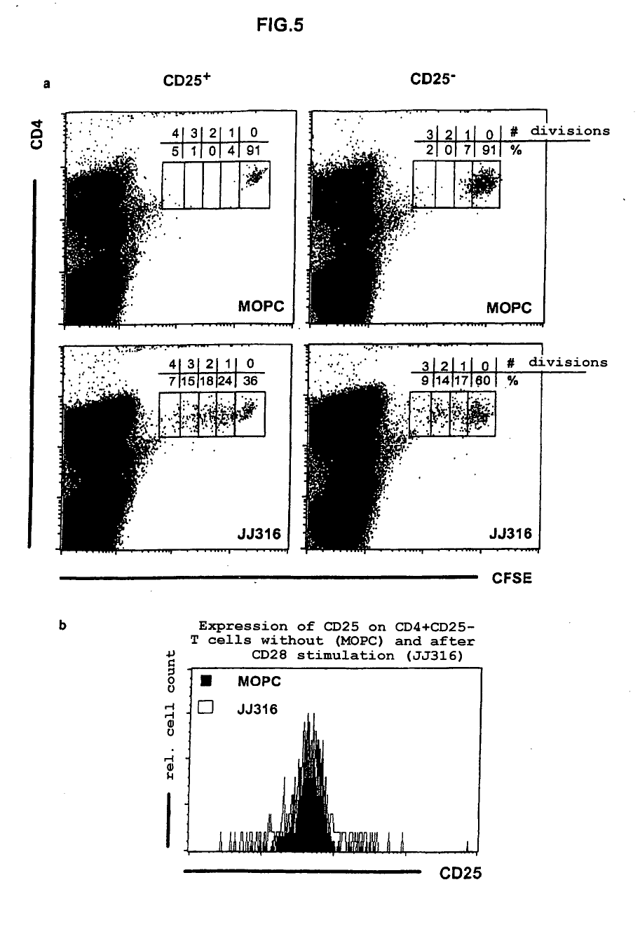

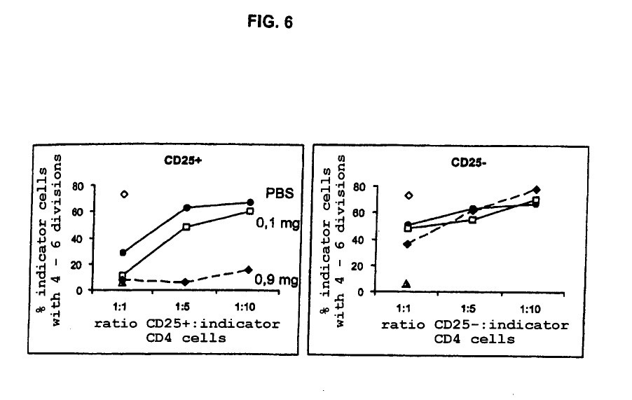

In healthy rats, with lowest-dosis administration of JJ316 (0.1 to 0.3 mg/animal), a stronger expansion of regulatory CD4+CD25+ T cells in lymph nodes and spleen can be found than of conventional CD4+CD25− T cells (see also FIG. 2). These regulatory T cells are phenotypically activated (FIG. 3), and a high portion thereof is in the cell cycle (FIGS. 4, 5). The suppressant properties of the Tregs, measured in vitro in a surrogate assay, are after administration of a lower dosis better than without pre-treatment (FIG. 6).

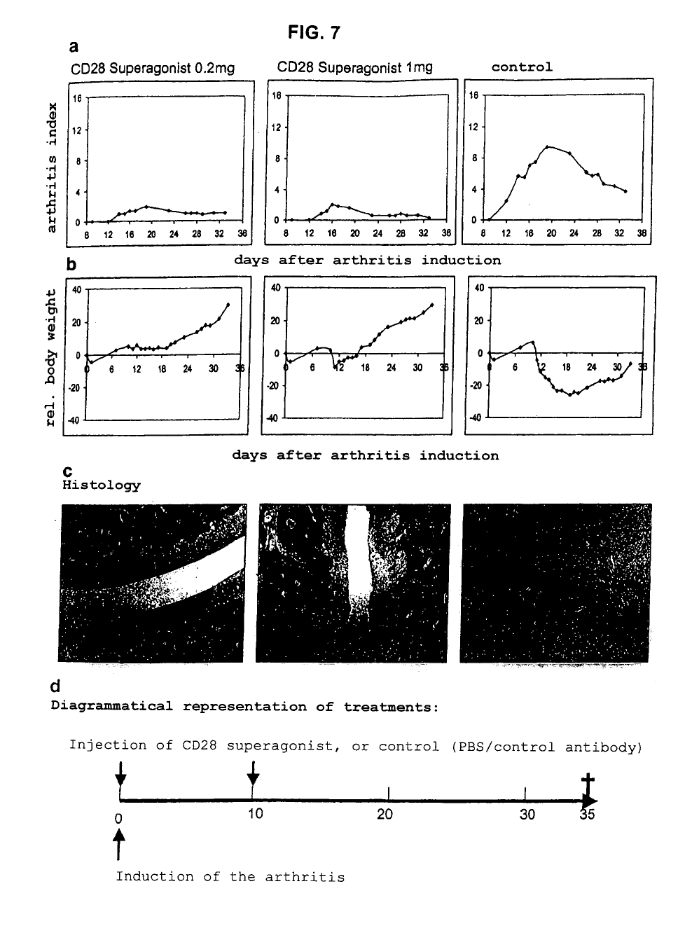

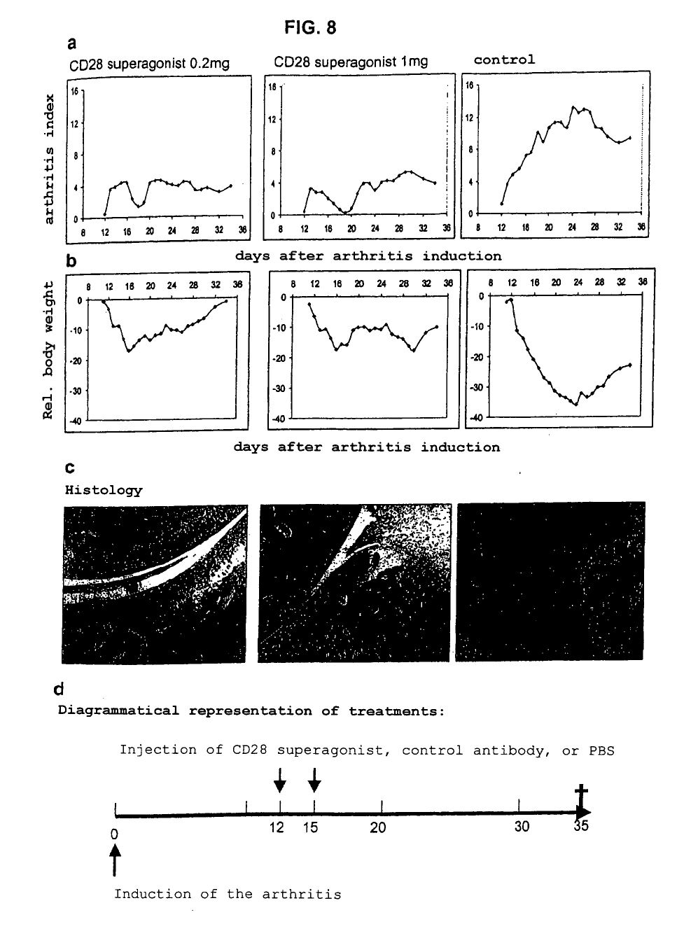

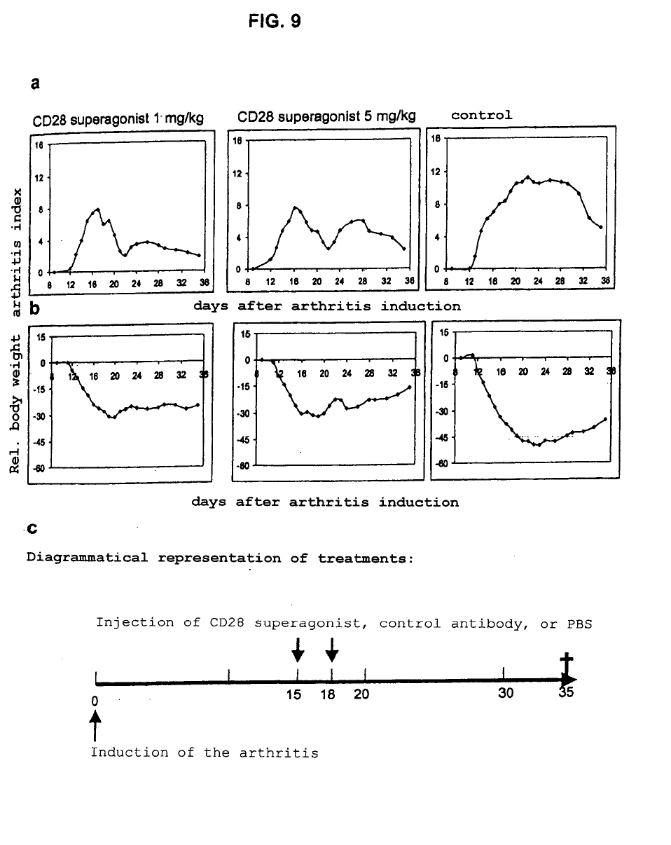

In the Applicant's previous patent application DE 197 22 888.7-41, the prophylactic properties of the antibody JJ316 for the prevention of the rat-adjuvant arthritis (as an animal model for the human RA) were mentioned. In the meantime, more comprehensive and better data were collected, which show that this effect cannot only be repeated with more animals and better controls (FIG. 7), but that the antibody administration is therapeutically effective even with an already established disease (FIGS. 8, 9)

All previous experiments concerning the anti-phlogistic properties of the rat CD28 superagonist in the animal models adjuvant arthritis (DE 197 22 888) and EAN (patent application PCT/DE03/00890) were performed as a routine with a as came to light—relatively high antibody dosis, namely 1 mg/animal. The new experiments show that at least in the adjuvant arthritis a 5 times lower dosis (0.2 mg/animal) has the same preventive or therapeutic effect (FIGS. 7-9).

The fact that Tregs are preferably expanded at the low doses, whilst a high dosis expands Tregs as well as “normal” T cells, has the implication that the indication fields “inflammatory autoimmune diseases” (effective mechanism of the antibody presumably via the activation of Tregs) and “immune reconstitution”, i.e. multiplication of all T cells, can be separated, i.e. inhibition of inflammation at low dosis and tolerated immune stimulation at high dosis.

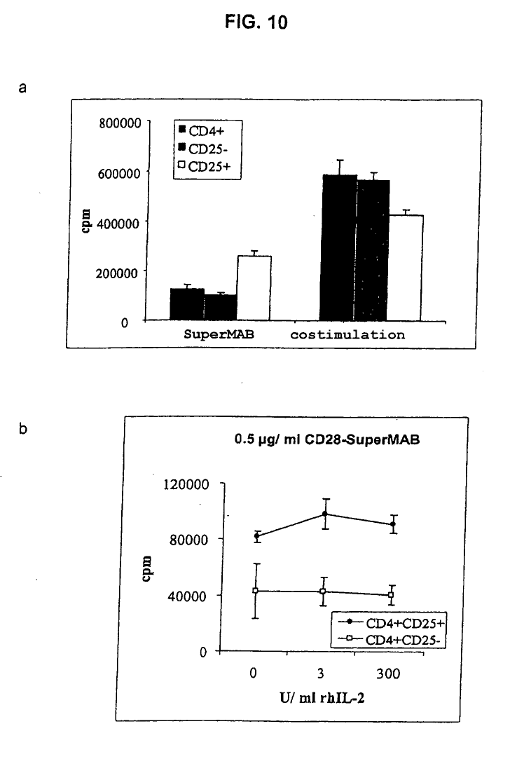

The results of FIG. 10 support older data of the patent application PCT/DE03/00890, namely that the human superagonist can drive Tregs in vitro to proliferation; FIG. 11 shows for the first time that the in vitro expanded CD4+CD25+ cells also have a suppressor function in an in vitro assay.

It is preferred when the MAB can be produced by that a non-human mammal is immunized with CD28 or a partial sequence thereof, in particular the C′-D loop and/or spatially adjacent sections, wherein cells are taken from the non-human mammal and hybridoma cells are produced from these cells, and wherein the thus obtained hybridoma cells are selected such that in their culture supernatant there are MABs superagonistically binding to CD28. But other usual methods for obtaining corresponding MABs, such as phage display or human Ig transgenic mice, can be used.

In principle, the antibodies according to the invention may be antibodies derived from the described antibodies, such as chimeric or humanized antibodies.

The mimetic compound is for instance obtainable in a screening method, wherein a prospective mimetic compound or a mixture of prospective mimetic compounds is subjected to a binding assay with CD28 or a partial sequence therefrom, in particular the C′-D loop and/or spatially adjacent sections, and wherein pharmaceutical substances binding to CD28 or to the partial sequence therefrom are selected, possibly followed by an assay for testing for superagonistic stimulation of several to all sub-groups of the T lymphocytes, wherein superagonistically stimulating pharmaceutical substances are selected.

The MAB is for instance obtainable from hybridoma cells, as filed under the DSM numbers DSM ACC2531 (MAB: 9D7 or 9D7G3H11) or DSM ACC2530 (MAB: 5.11A or 5.11A1C2H3). The MAB or the mimetic compound preferably comprises one or more of the sequences Seq. ID 9, 11, 13 and/or 15, or one or more of the sequences Seq. ID 10, 12, 14, and/or 16, or one or more of the sequences Seq. ID 18 and/or 19, or sequences being homologous hereto.

The invention also relates to a method or treatment plan for the treatment or prophylaxis of a disease, wherein either to a patient is administered a pharmaceutical composition comprising a CD28-specific superagonistic monoclonal antibody or a mimetic compound of the same and in a galenic preparation for a defined and suitable form of administration, for instance IV injection, or from a patient is taken a body liquid, in particular blood comprising T lymphocytes or precursor cells hereto, and the body liquid, possibly after a processing step, is reacted with a CD28-specific superagonistic monoclonal antibody or a mimetic compound of the same, and the thus treated body liquid is again administered to the patient, for instance by IV injection.

The administered daily dosis for the indication inhibition of inflammation may be below the first dosis limit of 1 mg/kg body weight, or for the indication immune stimulation above the second dosis limit of 2 mg/kg body weight.

An pharmaceutical substance according to the invention binds to CD28 or to a partial sequence therefrom. The partial sequence can for instance contain an amino acid sequence Seq.-ID 1 or 2-7 or 17, which lie at least partially in the region of the C′-D loop of CD28. To one of the sequences with val at the 5′ end, one or more amino acids of the sequence 8 may be connected in the order defined there. The loop is in the region with the sequence GNYSQQLQVYSKTGF. Mimetic compounds according to the invention can be identified in a screening method, a prospective mimetic compound or a mixture of prospective mimetic compounds being subjected to a binding assay with CD28 or a partial sequence therefrom, in particular the C′-D loop, and pharmaceutical substances binding to CD28 or to the partial sequence therefrom being selected, possibly followed by an assay for testing for superagonistic stimulation of several to all sub-groups of the T lymphocytes. In the case of a mixture it will be suitable to perform a deconvolution. Among the selected mimetic compounds so to speak a ranking according to the selectivity and/or affinity may be established, highly affinitive pharmaceutical substances being preferred. In addition to or in lieu of such a ranking, a ranking may be performed according to a quantification of the induction of the Tregs or according to the inhibition of the disease for instance in an animal test by using disease models.

An example of a pharmaceutical substance used according to the invention is a superagonistic CD28-specific MAB. It can for instance be produced by that a non-human mammal is immunized with CD28 or a peptide comprising a partial sequence therefrom, for instance as mentioned above or homologues hereto, cells being taken from the non-human mammal cells and hybridoma cells being produced from the cells, and the thus obtained hybridoma cells being selected such that in their culture supernatant there are MABs binding to CD28. A humanization can be performed with conventional methods. Suitable MABs can alternatively be produced by selecting B lymphocytes binding to the loop, and by cloning their expressed immunoglobulin genes. An isolation of suitable MABs from phages libraries is also possible.

In detail, this may be an MAB being obtainable from hybridoma cells, as filed under the DSM numbers DSM ACC2531 (MAB: 9D7 or 9D7G3H11) or DSM ACC2530 (MAB: 5.11A or 5.11A1C2H3). The MAB may comprise one or more of the sequences Seq. ID 9, 11, 13 and/or 15, or one or more of the sequences Seq. ID 10, 12, 14, 16, 18 and/or 19, or sequences being homologous hereto or being (partially) coded thereby. In Seq. ID 13 the nucleic acid sequence of the variable region of the heavy chain of an MAB 5.11A according to the invention is represented. Seq. ID 14 shows the peptide coded thereby. Seq. ID 15 shows the nucleic acid sequence of the variable region of the light chain of this MAB. Seq. ID 16 is the peptide coded hereby. In Seq. ID 9 the nucleic acid sequence of the variable region of the light chain of an MAB 9D7 according to the invention is represented. Seq. ID 10 shows the peptide coded hereby. Seq. ID 11 shows the nucleic acid sequence of the variable region of the heavy chain of this MAB. Seq. ID 12 is the peptide coded hereby. Seq. ID 18 and 19 show the amino acid sequences of the variable region of a humanized MAB 5.11A of the light chain and of the heavy chain, respectively.

The invention finally also relates to treatments, wherein to a person suffering from a disease caused by low regulator T cell counts or high T lymphocytes infiltration in organs or tissues, for instance the Guillain-Barré syndrome (GBS) and/or chronic demyelinating polyneuropathy (CDP), a pharmaceutical composition according to the invention is administered in a pharmacologically effective dose and in a galenic preparation suitable for the administration.

Definitions.

Monoclonal antibodies (MABs) are antibodies which are produced by hybrid cell lines (so-called hybridomas) which typically have been generated by fusion of a B cell of animal or human origin producing antibodies with a suitable myeloma tumor cell.

The amino acid sequence of human CD28 is known under accession No. NM_{—}006139.

The C′-D loop of CD28 comprises the amino acids 52 to 66 of the above CD28 sequence (for numbering see also Ostrov, D. A., et al.; Science (2000), 290:816-819). The term C′-D loop will in the following also include any partial sequences therefrom.

A loop or a binding site arranged therein is freely accessible, if for a defined binding partner for the binding site in the loop there is no steric hindrance by the sequences or molecules following to the loop.

Regulatory T cells are CD4+ T cells inhibiting in a mixture with naïve CD4+ T cells the activation thereof. Hereto belong in particular CD4+CD25+ T cells. Another feature of regulatory T cells is, compared to other T cells, a low expression of the high-molecular isoforms of CD45 (human: RA). For regulatory T cells, the constitutive expression of CD152 is typical. CD4+CD8-SP thymocytes are one of the essential sources for regulatory T cells. For a further characterization of regulatory T cells, reference is made to the document K. J. Maloy et al., Nature Immunology, Vol. 2, No. 9, pages 816 ff., 2001.

The induction of regulatory T cells is the increase of the metabolic activity, enlargement of the cell volume, synthesis of immunologically important molecules and beginning of the cell division (proliferation) upon an external stimulation. As a result, after the induction there are more regulatory T cells than before.

Homology is an at least 70%, preferably at least 80%, most preferably at least 90% sequence identity on a protein level, a homologous protein or peptide binding a defined binding partner with at least identical affinity. Deviations in the sequence may be deletions, substitutions, insertions and elongations.

A mimetic compound is a natural or synthetic chemical structure behaving in a defined binding assay as a defined MAB mimicrying the mimetic compound.

The term MABs comprises, in addition to structures of the conventional Fab/Fc type, also structures exclusively consisting of the Fab fragment. It is also possible to use the variable region only, the fragment of the heavy chains being connected with the fragment of the light chain in a suitable manner, for instance also by means of synthetic bridge molecules. The term antibody also comprises (possibly complete) chimeric and humanized antibodies.

Superagonistic stimulation of the proliferation of CD28-specific T cells means that no costimulation, i.e. no further binding event in addition to a binding of an MAB or of a mimetic compound is necessary for the stimulation or inhibition of the proliferation, and that several to all sub-groups of the T lymphocytes are activated by such superagonistic CD28-specific MABs.

A lower dosis limit can individually be found out for every pharmaceutical substance according to the invention by that first the cell counts of CD25+ and CD25− cells per volume unit in the blood of an organism are determined, that then the pharmaceutical substance is administered in respectively increasing dosis to the organism or an identical organism, and for instance 3 to 20 days after the administration of a dosis the cell counts of CD4+CD25+ and CD4+CD25− cells per volume unit bloods are again determined, and the obtained cell counts (ordinate) against dosis (abscissa) are entered into a diagram and connected so to form curves, for instance by a polynomial fit, and the first derivative of the curves at the dosis is formed and the lower dosis limit is determined as that dosis, where the first derivative of the curve for CD4+CD25+ has the same value as the first derivative of the curve for CD4+CD25−, and that within a tolerance deviation of less than 50% (related to the value of the derivative for CD4+CD25+), preferably less than 30%, most preferably less than 10%. This value can simultaneously be the second dosis limit. It may however also be provided that the lower dosis limit is that value, where the value of the derivative for CD4+CD25− changes the sign from negative to positive. The upper dosis limit can then be determined as already mentioned above. Alternatively, the lower dosis limit may be defined by that the relative increase in said period of time of the CD4+CD25+ cells in the organism is at least by a factor two higher than the relative increase of the CD4+CD25− cells (example: quadruplication of the CD4+CD25+ cells at duplication of the CD4+CD25− cells). The upper dosis limit could then be defined as the same relative increase of both cell types. For man, typical values for the lower dosis limit will be less than 1 mg/kg body weight and day. The upper dosis limit will be 2 mg/kg body weight and day.

Typical diseases of the indication inhibition of inflammation are rheumatoid arthritis, multiple sclerosis, insulin-depending (type 1) diabetes mellitus, Crohn's disease, psoriasis, Guillain-Barré syndrome (GBS), graft-versus-host disease (GVHD).

Typical diseases of the indication immune stimulation are chronic lymphocytic leukemia of the B cell type (B-CLL), acute lymphoblastic leukemia (A-LL), T lymphopenia after chemo- or radio therapy, as performed for instance with various solid tumors, such as lung carcinoma or mamma carcinoma, HIV infection, HTLV infection.

Examples of Execution.

In the following, the invention and the basic findings are explained in more detail.

FIG. 1 shows the long-term expansion of regulatory CD4+CD25+ T cells in vitro. It has been shown by Lin et al. (Lin and Hunig, 2003) that the superagonistic anti-rat CD28 MAB JJ316 is capable to very efficiently activate regulatory CD4+CD25+ CTLA-4+ T cells in short-term cell culture. The question came up whether another result is the long-term expansion of Tregs. FIG. 1a shows that this antibody also allows a long-term expansion of the CD4+CD25+ T cells: 6×10ˆ4 CD4+CD25+ and CD4+ CD25− T cells were isolated from a normal Lewis rat and cultivated in presence of JJ316 (5 μg/ml) and interleukin-2 (300 U/ml) in microtiter plates (96-well, 12-well or 6-well plates) at 37° C., and the cells were weekly supplied with fresh reagents. Also in this long-term culture, a preferential expansion of the CD4+CD25+ T cells compared to the CD4+CD25− T cells can be observed. Beginning from the initial cell count (FIG. 1a shows relative values with regard to the cell count; the absolute cell count on day 0 was taken as 1), the CD4+CD25+ T cells could by multiplied by the factor 6.1×10ˆ7. For the CD4+CD25− T cells, this value is only 6.7×10ˆ5 (FIG. 1a).

The functional properties of regulatory T cells in vitro are characterized by that they are capable to suppress the proliferation of conventional T cells. By means of an in vitro test system, it was investigated whether the CD4+CD25+ T cells expanded by JJ316 could maintain this function (FIG. 1b): 5×10ˆ4 conventional CD4+ T cells serving as indicator cells in this test were cultivated either alone or together with increasing amounts (5×10ˆ3, 1×10ˆ4 or 5×10ˆ4 cells) of the CD4+CD25+ or CD4+ CD25− T cells of day 78 of the long-term culture (FIG. 1a) for three days in 96-well microtiter plates in 0.2 μl. The stimulation of the cells was made by addition of the lectin ConA (2 μl/ml) and antigen-presenting cells (5×10ˆ4). The cell proliferation in the culture was measured by means of integration of 3H thymidine during the last 16 hours of the assay, as described in the literature. The polyclonal stimulation of CD4+ indicator cells by ConA (column 1) could significantly be reduced by addition of the expanded CD4+CD25+ T cells (columns 2-4). In contrast, the expanded CD4+CD25− T cells (column 5) showed no suppressant functions, as expected. In contrast to the CD4+CD25− T cells (column 7), the JJ316-stimulated CD4+CD25+ T cells (column 6) could be stimulated by ConA to a very low degree only, thereby indicating that the values shown in columns 2-4 nearly exclusively reflect the proliferation of the indicator cells. This result shows that the in vitro expanded regulatory T cells could maintain their functional—i.e. suppressant—properties.

FIG. 2 shows the preferential multiplication of regulatory CD4+CD25+ T cells after in vivo application of JJ316 in normal Lewis rats. After the above in vitro experiments had shown that superagonistic CD28-specific MABs can induce a preferential expansion of CD4+CD25+ T cells while maintaining their functional properties, it was investigated whether similar effects can be observed also after injection of these antibodies in the test animal: normal adult Lewis rats were JJ 316 IV applied in different doses (0.1; 0.3 and 0.9 and 2.7 mg/animal). After 3 gays, spleen and lymph nodes were removed, and the lymphocyte subpopulations of these organs were analyzed in the FACS (measured parameters: CD5, CD4 and CD25). The relative values of the CD4+CD25+ and the CD4+CD25− T cells determined by the FACS analysis were multiplied with the total cell count/organ, and the thus obtained number of the two populations were recorded as a function of the applied dosis. In FIG. 2, the results of three independent experiments are represented. As the slope of the curve in particular in the lower dosis range and in particular in the lymph nodes, an increased expansion of CD4+CD25+ compared to CD4+CD25− T cells can be observed after in vivo application of JJ316. This experiment clearly shows that superagonistic CD28-specific monoclonal antibodies multiply mainly regulatory T cells also in vivo already at low doses and can thus potentially re-establish the disturbed balance for many autoimmune diseases between regulatory T cells and autoreactive T cells.

FIG. 3 shows the phenotype of JJ316-expanded cells. In the previous experiment, the absolute cell counts of CD4+CD25+ and CD4+CD25− T cells were determined after in vivo applications, and it was shown for the lower dosis JJ316, the number of the CD4+CD25+ cells in the lymph node increases to a higher degree than the number of the CD4+CD25− cells. Then, it was of interest to determine the activation or differentiation state of the CD4+CD25+ and CD4+CD25− T cells by means of flow cytometric analysis of the expression of the T cell markers OX40, OX6 (MHC class II) and CD45RC. 0.2 or 1 mg JJ316 were IV injected into male rats. Four days later, i.e. at a time near to the maximum expansion of the CD4+CD25+ cells (Lin and Hunig, 2003), lymph node cells were isolated, and the cell surface expression of OX40, OX6 (MHC class II) and CD45RC was determined. In FIG. 3 is shown the relative cell sizes (x axis) relative to the activation markers (y axis) among the CD4+CD25− and CD4+CD25+ cells of the control group (top), the low-dosis group (center) and the high-dosis group (bottom). High expression of OX40 or OX6 and low expression of CD45RC and increase of the relative cell size in the so-called forward scatter, fsc, characterizes activated cells. Therefore, CD4+CD25− as well as CD4+CD25+ cells were activated by both doses of JJ316. For the conventional CD4+CD25− cells, the activation seemed to be stronger for the higher dosis than for the lower dosis (increase in particular of the fsc and reduction of CD45RC, however lower increase of OX6). The regulatory CD4+CD25+ cells had as expected in the control group already a high portion of pre-activated cells (low portion of CD45RC+ cells), which was even increased by both doses (see in particular fsc, OX40). The higher expression and the larger fsc indicate that the activation in the high-dosis group was stronger than in the low-dosis group.

FIG. 4 shows in vivo proliferation of CD4+CD25+ and CD4+CD25− T cells after administration of JJ316. FIG. 3 showed that conventional CD4+CD25− cells as well as regulatory CD4+CD25+ cells were phenotypically activated in vivo by both doses of JJ316. Subsequently, by means of the proliferation marker Ki67, it was measured whether the activation also correlates with a cell division. The test approach was identical to that of FIG. 3. The portion of the CD4+CD25+ or CD4+CD25− cells being in the cell cycle was determined by extracellular staining on CD4 and CD25 expression as well as by intracellular staining on Ki-67 expression (characterizes cells, which are in the S or G2/M phase of the cell division and thus actively proliferate). In FIG. 4 can be seen that with regard to the control group, the treatment with JJ316 induces in both populations a strong Ki-67 expression and thus active DNA synthesis, which was slightly stronger for the high dosis than for the low dosis. As a summary, the experiments show that by IV application of 0.2 mg or 1 mg JJ316, a profound phenotypic activation and multiplication of Tregs in rats is induced.

FIG. 5 shows that JJ316 expands in vivo CD4+CD25+ T cells, without inducing the expression of CD25 to before CD25-negative (conventional) T cells. The prior experiments could not provide a direct proof that the increased absolute number of the CD4+CD25+ T cells after the in vivo application of JJ316 is the consequence of the expansion of these cells and does not rely on that originally CD25-negative T cells express after activation of this protein as an “activation marker” on the surface (as this can be observed after in vitro activation of T cells). For clarifying this question, CD4+CD25+/CD4+CD25− T cells from the spleen and lymph nodes of normal rats were isolated, and the two populations were labeled with the dye carboxyfluorescein succinimidyl ester (CFSE, 2.5 μM). CFSE is incorporated in the cytoplasm of the cell and distributed after every cell division by 50% each on the daughter cells. By means of FACS analysis, the CFSE content of a cell population can be analyzed and thus the number of the obtained cell divisions can be determined. 5×10ˆ6 CD4+CD25− T cells and 1×10ˆ6 CD4+CD25+ CFSE-labeled T cells were transferred into respectively different receiver animals, into which one day later either a control antibody (MOPC) or JJ316 each in the low dosis of 0.2 mg/animal were IV applied. After two days, spleen and lymph nodes of these animals were removed, the T cells were purified by means of nylon wool, and their CFSE content were analyzed in the FACS. FIG. 5a shows that the application of JJ316 not only induces a dilution of the CFSE content of the transferred CD4+CD25−, but also of the CD4+CD25+ T cells. This result thus supplies direct proof that JJ316 can induce in vivo an expansion of CD4+CD25+ T cells. In this experiment, too, we could observe a preferential expansion of the CD4+CD25+ T cells by JJ316, since the average number of cell divisions is higher (1.3) for the CD4+CD25+ T cells than for the CD4+CD25− T cells (0.7). This finding that CD25 is not induced in vivo in CD4+CD25− T cells by JJ316 stimulation (FIG. 5b), further indicates that the increased number of CD4+CD25+ T cells is exclusively a consequence of the expansion of CD4+CD25+ “mother” cells.

FIG. 6 shows the functional characterization of regulatory CD4+CD25+ T cells expanded in vivo by JJ316. It is of decisive importance whether the CD4+CD25+ T cells expanded in vivo by JJ316 maintain their suppressant properties. For clarifying this question, JJ316 was applied in different doses (0.1 mg and 0.9 mg) into normal rats, as described above. After three days CD4+CD25+/CD4+CD25− T cells were isolated and tested in vitro for their suppressant potential: it was investigated how strongly the two populations are capable to inhibit in vitro the proliferation of CD4+ T cells activated by conventional costimulation (=indicator cells). The expanded CD4+CD25+ or CD4+ CD25− T cells were cocultivated in this test in different ratios (1:1, 1:5 and 1:10) with CFSE-labeled CD4+ indicator cells in presence of monoclonal antibodies with specificity for the T cell receptor (R73; 1 μg/ml) and CD28 (JJ319; 0.5 μg/ml) for five days, and then the CFSE content of the indicator cells (as a measure of the occurred cell divisions) was analyzed in the FACS. Whilst in absence of the CD4+CD25+ T cells still approx. 75% of the indicator cells could undergo 4-6 cell divisions (FIG. 6, open rhombus), this portion was distinctly reduced after addition of the expanded CD4+CD25+ T cells, and that the stronger, the higher was the dosis of JJ316 previously applied in the animals (FIG. 6, closed circles=PBS control, open squares=0.1 mg JJ316, closed rhombi=0.9 mg JJ316). On the other hand, such an effect could not be observed for the CD4+CD25− T cells. After in vivo application of superagonistic CD28-specific monoclonal antibodies, thus not only a purely numerical multiplication of regulatory T cells is induced, rather the suppressant properties of these cells can even be increased with an increasing antibody dosis.

FIG. 7 shows the preventive effect of JJ316 on the development of adjuvant arthritis. The previous results show that JJ316 induces in vivo and in vitro an expansion of functional regulatory CD4+CD25+ cells. The important question was open whether this expansion also correlates with an anti-inflammatory effect of the antibody, and if that is the case whether the Tregs are really causally involved in the mediation of such an effect. Whilst the following results indicate a clearly positive answer to the first question, the question for the mechanism of the inflammation prophylaxis and therapy, as shown by FIGS. 7-9, by CD28 superagonists is not yet fully answered. For the investigation of the anti-phlogistic effect of JJ316, the animal model of the adjuvant arthritis in Lewis rats (Barnes et al., 1998) was used. For the disease induction, 6-8 weeks old male rats (200 to 250 g) were intradermally injected (day 0) at the base of the tail with 0.1 ml Mycobacterium tuberculosis (heat-killed) in IFA (incomplete Freund's adjuvant, 5 mg/ml). In order to investigate in the first group the prophylactic effect of JJ316, six rats each were intravenously applied on day 0 and day 10 0.2 or 1 mg JJ316. As a control group served six rats, which were treated on day 0 and day 10 with 0 mg, 0.2 mg or 1 mg irrelevant control antibodies of the same isotype (mouse IgG1) (shown is here as in FIGS. 8 and 9 a group treated with 1 mg control antibody; identical results were obtained in the other control groups). The development of arthritis was continuously examined by objective evaluation of the joint inflammations, represented as the arthritis index consisting of a combined determination of reddening, swelling and stiffening of the joints, and the surrogate parameter “weight measurement”. The experiment was terminated on day 35 by killing the animals. The back paws were fixed in a 10% formalin solution for 72 hours. After decalcification in 10% EDTA solution (24 weeks), the joint tissue was histologically processed as described by (Barnes et al., 1998) by staining with safranin O, fast green and hematoxylin and analyzed with regard to cartilage destruction and cellular infiltrates. In FIG. 7a, b the result of the preventive treatment of six animals each are summarized in their mean values. Therefrom results that in the control animals an arthritis occurs beginning on day 9 with a maximum after approx. 3 weeks (top right), which is characterized by an accompanying weight loss (center right). By treatment with a low dosis of 0.2 mg JJ316 (left column), same as with a high dosis of 1 mg JJ316 (central column), the development of the arthritis as well as the weight loss could nearly completely be prevented. It is remarkable that the prophylactic effect of the low dosis is at least as good as the effect of the 5-fold higher dosis. The equivalent protection from arthritis by a high- and low-dosis JJ316 treatment could also be shown on a histologic level: whilst for control animals, the joints of the back paws are characterized by massive cellular infiltrates as a sign of an inflammation reaction (FIG. 7c, right), the joint gaps of the JJ316-treated animals have a normal architecture (FIG. 7c, center and left). The treatment scheme is summarized in FIG. 7d. To sum up, these results show that high- and low-dosis JJ316 treatment at the time of the arthritis induction has an equally strong preventive effect on the development of the arthritis.

FIG. 8 shows the therapeutic effect of JJ316-treatment on adjuvant arthritis (early intervention). Then the important question was examined whether JJ316 treatment has not only a preventive, but also a curative effect on the disease progress. This is particularly relevant, since for the clinical treatment of the RA with the human CD28-SuperMAB no prophylaxis, but exclusively a therapy is possible. In the experiment shown in FIG. 8, arthritis was induced for six animals each per group, as described in FIG. 7. On day 12 and 15 after disease induction, i.e. at the time of a very mild development of the symptoms (average arthritis index 0.5) and three days later, the animals were treated with 0.2 mg JJ316, 1.0 mg JJ316 or an irrelevant control antibody. As is shown in FIG. 8a, for the JJ316-treated animals there was, after an initial development of the symptoms, a stabilization of the disease (max. arthritis index 4-5), and in the meantime a distinct reduction of the symptoms and a stabilization of the weight. Again the therapeutic effect was very similar for high and low dosis of JJ316. For the control-treated animals, there was however a fulminant arthritis with a maximum arthritis index of >12 and a progressive weight loss (FIG. 8a, b). Equally, the JJ316 treatment led with both doses to a maintenance of the normal architecture of the joint tissue, which in the control group showed clear signs of inflammatory infiltrates and morphologic destruction (FIG. 8c). To sum up, with an early therapeutic intervention a low dosis of JJ316 same as a high dosis of JJ316 will prevent the clinical development of a serious arthritis and the destruction of tissue.

FIG. 9 shows the therapeutic effect of JJ316 treatment on adjuvant arthritis (late intervention). Subsequently, it was investigated whether an arthritis therapy is possible by CD28 superagonist administration even with clearly developed disease symptoms. The experimental approach corresponded to that as described in FIG. 8, however animals, which have become already clearly diseased, were treated on day 15, i.e. at a time when the arthritis development was given the mark 6.5 on average, and on day 18 with JJ316 (0.2 and 1.0 mg/animal) or with a control antibody (FIG. 9). For the JJ316-treated animals, a distinct reduction of the disease symptoms approx. beginning on day 16 was found. This effect was even more developed for a low JJ316 dosis than for the high dosis. Further, the weight was stable for the experimental group, whereas the control group has a progressing weight loss (FIG. 8b). To sum up, even in this extremely complicated late pharmacological intervention, a low JJ316 dosis had an at least equally clear curative effect on the course of the arthritis in the rat model as a high JJ316 dosis.

FIG. 10 shows the preferential expansion of human CD4+CD25+ T cells by means of SuperMAB. As already described in earlier documents, superagonistic monoclonal antibodies with specificity for human CD28 (=SuperMAB) were generated. Here, too, it was important to optimize the stimulation and culture conditions, in order to guarantee an efficient expansion of human CD4+CD25+ T cells by means of SuperMAB: from voluntary healthy donors, T lymphocytes were prepared and separated into “conventional” CD4+CD25− and regulatory CD4+CD25+ T cells (purity as a routine approx. 90%). 5×10ˆ4 each of both populations were cultivated in presence of cross-linked (by means of 40 μg/ml anti-mouse Ig antiserum) SuperMAB (10 μg/ml) or by classic costimulation by means of cross-linked monoclonal antibodies with specificity for CD3 (10 μg/ml) and CD28 (10 μg/ml) in 96-well microtiter plates. After three days, the proliferation was tested by means of incorporation of 3H thymidine. In a similar way as described for JJ316, CD4+CD25+ T cells were also driven by SuperMAB to a stronger degree into proliferation than conventional CD4+CD25− T cells (FIG. 10a). This result was reproduced in a following experiment, wherein simultaneously the effect of the growth factor interleukin-2 was investigated for the stimulation of the CD4 subpopulations CD4+ CD25+ and CD4+ CD25− by means of SuperMAB. As is shown in FIG. 10b, IL-2 did not have any decisive influence in this experiment on the proliferation of phenotypically regulatory CD4+CD25+ or conventional CD4+CD25− T cells. These in vitro experiments show that SuperMAB is also capable to primarily multiply regulatory T cells.

FIG. 11 verifies that CD4+CD25+ T cells expanded in vitro by SuperMAB are regulatory T cells. As already explained for the system rat, here the question was, too, whether the CD4+CD25+ T cells expanded by SuperMAB could maintain their functional properties, namely the capability to suppress the proliferation of conventional T cells. For this purpose, CD4+CD25+ and CD4+CD25− T cells of healthy donors were isolated and cultivated for 12 days in presence of SuperMAB (0.4 μg/ml) and interleukin-2 (100 U/ml). In this experiment, too, the CD4+CD25+ T cells could be driven into the proliferation better than the CD4+CD25− T cells (FIG. 11a). In a second culture, then the cells expanded by SuperMAB were tested for their suppressant potential. Analogously to the experiments projected for the rat, it was investigated whether they are capable to inhibit the proliferation of syngeneic and CFSE-labeled CD4+ T cells activated by conventional costimulation (=indicator cells): The expanded CD4+CD25+ and CD4+CD25− T cells were cocultivated for five days in different ratios (1:1, 1:5 and 1:10) with CFSE-labeled CD4+ indicator cells in presence of monoclonal antibodies with a specificity for CD3 (0.1 μg/ml) and CD28 (0.05 μg/ml), and then the CFSE content of the indicator cells (as a measure of the achieved cell divisions) was analyzed in the FACS. Whilst in absence of the CD4+CD25+ T cells still approx. 30% of the indicator cells could undergo 3-5 cell divisions (FIG. 11b, open rhombus), this portion was clearly reduced after addition of the SuperMAB-expanded CD4+CD25+ T cells (closed circles). A low suppressant effect showed the expanded CD4+CD25− T cells, too (open squares), which possibly is based on that a few CD25+ T cells were present within the population of CD25− T cells and have transferred their suppressant properties to a part of the CD25− T cells during the SuperMAB-driven expansion phase. Alternatively, a stimulation of CD25-regulatory T cells by SuperMAB is also imaginable. This experiment shows that—as has already been shown for the system rat—SuperMAB-expanded human CD4+CD25+ T cells also maintain their functional properties.

1. A pharmaceutical composition for the treatment or prophylaxis of autoimmune-caused inflammatory diseases or for the immune reconstitution comprising a CD28-specific superagonistic monoclonal antibody (MAB) or a mimetic compound of the same, wherein the pharmaceutical composition is prepared such that the dosage of the MAB to be administered is below a defined first dosage limit, if the treatment or prophylaxis of an autoimmune-caused inflammatory disease is indicated, and that the dosage of the MAB to be administered is above a defined second dosage limit, if an immune reconstitution is indicated.

2. The composition of claim 1, wherein the MAB is prepared from a non-human mammal immunized with CD28 or a partial sequence thereof.

3. The composition of claim 1, wherein the mimetic compound is obtainable in a screening method comprising the following steps: a prospective mimetic compound or a mixture of prospective mimetic compounds is subjected to a binding assay with CD28 or a partial sequence therefrom, and pharmaceutical substances binding to CD28 or to the partial sequence thereof are selected.

4. The composition of claims 1 or 2, wherein the MAB is obtainable from hybridoma cells, as filed under the DSM numbers DSM ACC2531 (MAB: 9D7 or 9D7G3H11) or DSM ACC2530 (MAB: 5.11A or 5.11A1C2H3).

5. The composition of claims 1 or 2, wherein the MAB or the mimetic compound comprises one or more of the sequences Seq. ID 9, 11, 13 or 15, or one or more of the sequences Seq. ID 10, 12, 14, or 16, or one or more of the sequences Seq. ID 18 or 19, or 20 or 21, or sequences being homologous hereto.

6. A method or treatment plan for the treatment or prophylaxis of a disease according to claim 1, wherein
to a patient is administered a pharmaceutical composition comprising a CD28-specific superagonistic monoclonal antibody or a mimetic compound of the same and in a galenic preparation for a defined and suitable form of administration comprising intraveneous injection.

7. A method or a treatment plan according to claim 6, wherein the administered dosage for the indication for inhibition of inflammation is below the first dosage limit of 0.1 to 10 mg/kg body weight daily, or wherein the administered dosage for the indication immune stimulation is above the second dosage limit of 0.1 to 10 mg/kg body weight daily, the second dosage limit always being equal to or greater than the first dosage limit.

8. The composition of claim 1, wherein the partial sequence of CD28 comprises a C′-D loop or spacially adjacent sections of CD28.

9. The composition of claim 2, wherein the MAB is further prepared by taking cells from a non-human mammal and producing hybridoma cells from the non-human mammal cells, wherein the hybridoma cells are selected such that their culture supernatant contains MABs which superagnonistically bind to CD 28.

10. The composition of claim 1, wherein the partial sequence of CD28 comprises a C′-D loop, or spacially adjacent sections of CD28.

11. The composition of claim 3, further comprising the following step:
the pharmaceutical substances binding to CD28 or to the partial sequence thereof is assayed to test for superagonistic stimulation of several to all sub-groups of the T lymphocytes, wherein superagonistically stimulating pharmaceutical substances are selected.

12. A method or treatment plan for the treatment or prophylaxis of a disease according to claim 1, wherein from a patient is taken a body liquid, comprising blood comprising T lymphocytes or precursor cells hereto, and the body liquid is reacted with a CD28-specific superagonistic monoclonal antibody or a mimetic compound of the same, and the thus treated body liquid is again administered to the patient by a suitable form of administration comprising intraveneous injection


SEQUENCE LISTING (19 sequences)

SEQ ID NO: 1 - protein, 6 aa, Artificial
  human CD28 partial sequence
VYSKTG

SEQ ID NO: 2 - protein, 4 aa, Artificial
  human CD28 partial sequence
YSKT

SEQ ID NO: 3 - protein, 5 aa, Artificial
  human CD28 partial sequence
YSKTG

SEQ ID NO: 4 - protein, 5 aa, Artificial
  human CD28 partial sequence
VYSKT

SEQ ID NO: 5 - protein, 6 aa, Artificial
  human CD28 partial sequence
YSKTGF

SEQ ID NO: 6 - protein, 7 aa, Artificial
  human CD28 partial sequence
VYSKTGF

SEQ ID NO: 7 - protein, 5 aa, Artificial
  human CD28 partial sequence
SKTGF

SEQ ID NO: 8 - protein, 8 aa, Artificial
  human CD28 partial sequence
GNYSQQLQ

SEQ ID NO: 9 - DNA, 321 bp, Artificial
  monoclonal antibody 9D7 partial sequence
gatatccagacgacacagactacatcctcccgttctgcctctctgggagacagagtcaccatcagttgcagggcaggtcaggacattagtaattatttaaactggtatcagcagaaaccagatggaactgttaagctcctgatctactacacatcaagattacactcaggagtcccatcaaggttcagtggcagtgggtctggaacagattattctctcaccattagcaacctggagcaagaagatattgccacttacttttgccaacagggtcatacgcttccgtggacgttcggtggaggcaccaagctggaaatcaaa

SEQ ID NO: 10 - protein, 107 aa, Artificial
  monoclonal antibody 9D7 partial sequence
DIQTTQTTSSLSASLGDRVTISCRAGQDISNYLNWYQQKPDGTVKLLIYYTSRLHSGVPSRFSGSGSGTDYSLTISNLEQEDIATYFCQQGHTLPWTFGGGTKLEIK

SEQ ID NO: 11 - DNA, 363 bp, Artificial
  monoclonal antibody 9D7 partial sequence
gatgtgcagcttcaggagtcgggacctggcctggtgaaaccttctcagtctctgtccctcacctgcactgtcactggctactcaatcaccagtgattatgcctggaactggatccggcagtttccaggaaacaaactggagtggatgggctacataagatacagtggtagtactagctacaatccatctctcaaaagtcgaatctctatcactcgagacacatccaagaaccagttcttcctgcagttgaattctgtgactactgaggacacagccacatattactgtgcaagagattggccgcgaccgagctactggtacttcgatgtctggggcgcagggaccacggtcaccgtctcctca

SEQ ID NO: 12 - protein, 121 aa, Artificial
  monoclonal antibody 9D7 partial sequence
DVQLQESGPGLVKPSQSLSLTCTVTGYSITSDYAWNWIRQFPGNKLEWMGYIRYSGSTSYNPSLKSRISITRDTSKNQFFLQLNSVTTEDTATYYCARDWPRPSYWYFDVWGAGTTVTVSS

SEQ ID NO: 13 - DNA, 360 bp, Artificial
  monoclonal antibody 5.11A partial sequence
caggtccaactgcagcagtccggacctgagctggtgaagccggggacttcagtgaggatttcctgcgaggcttctggctacaccttcacaagctactatatacactgggtgaaacagaggcctggacagggacttgagtggattggatgtatttatcctggaaatgtcaatactaactataatgagaagttcaaggacaaggccacactgattgtagacacatcctccaacactgcctacatgcagctcagcagaatgacctctgaggactctgcggtctatttctgtacaagatcacactacggcctcgactggaacttcgatgtctggggcgcagggaccacggtcaccgtctcctca

SEQ ID NO: 14 - protein, 120 aa, Artificial
  monoclonal antibody 5.11A partial sequence
QVQLQQSGPELVKPGTSVRISCEASGYTFTSYYIHWVKQRPGQGLEWIGCIYPGNVNTNYNEKFKDKATLIVDTSSNTAYMQLSRMTSEDSAVYFCTRSHYGLDWNFDVWGAGTTVTVSS

SEQ ID NO: 15 - DNA, 321 bp, Artificial
  monoclonal antibody 5.11A partial sequence
gacatccagatgaaccagtctccatccagtctgtctgcatcccttggagacacaattaccatcacttgccatgccagtcaaaacatttatgtttggttaaactggtaccagcagaaaccaggaaatattcctaaactcttgatctataaggcttccaacctgcacacaggcgtcccatcaaggtttagtggcagtggatctggaacaggcttcacattaaccatcagcagcctgcagcctgaagacattgccacttactactgtcaacagggtcaaacttatccgtacacgttcggaggggggaccaagctggaaataaaa

SEQ ID NO: 16 - protein, 107 aa, Artificial
  monoclonal antibody 5.11A partial sequence
DIQMNQSPSSLSASLGDTITITCHASQNIYVWLNWYQQKPGNIPKLLIYKASNLHTGVPSRFSGSGSGTGFTLTISSLQPEDIATYYCQQGQTYPYTFGGGTKLEIK

SEQ ID NO: 17 - protein, 15 aa, Artificial
  human CD28 partial sequence
GNYSQQLQVYSKTGF

SEQ ID NO: 18 - protein, 107 aa, Artificial
  monoclonal antibody 5.11A partial sequence
DIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIK

SEQ ID NO: 19 - protein, 120 aa, Artificial
  monoclonal antibody 5.11A partial sequence
QVQLVQSGAEVKKPGASVKVSCKASGYTFTSYYIHWVRQAPGQGLEWIGCIYPGNVNTNYNEKFKDRATLTVDTSISTAYMELSRLRSDDTAVYFCTRSHYGLDWNFDVWGQGTTVTVSS